(19) United States
(12) Patent Application Publication
(10) Pub. No.: US 2022/0130538 A1
(43) Pub. Date: Apr. 28, 2022

(54) Method For Assessing Acute Kidney Injury And Acute Kidney Injury Assessment System

(71) Applicant: China Medical University, Taichung City (TW)

(72) Inventors: Chin-Chi Kuo, Taichung City (TW); Hung-Chieh Yeh, Taichung City (TW)

(73) Assignee: China Medical University, Taichung City (TW)

(21) Appl. No.: 17/105,714

(22) Filed: Nov. 27, 2020

(30) Foreign Application Priority Data
Oct. 22, 2020 (TW) 109136737

(51) Int. Cl. G16H 50/20; G16H 50/30; G16H 10/60; G16H 10/40; G16H 50/70; G16H 70/40; G16H 15/00; G16H 20/10; G06N 20/00

(52) U.S. Cl. CPC G16H 50/20; G16H 50/30; G16H 10/60; G16H 10/40; G06N 20/00; G16H 70/40; G16H 15/00; G16H 20/10; G16H 50/70

(57) ABSTRACT

A method for assessing acute kidney injury includes following steps. An acute kidney injury assessing date of a subject is provided. A testing kidney function diagnostic dataset is provided, wherein the testing kidney function diagnostic dataset includes a plurality of serum creatinine concentration data and a plurality of glomerular filtration rate data, and a recording date of each of the serum creatinine concentration data and a recording date of each of the glomerular filtration rate data is on 0 to 180 days before the acute kidney injury assessing date. A preprocessing step is performed. A first classifying step is performed, wherein a fluctuation value of serum creatinine concentration is classified according to a first threshold or a fluctuation value of eGFR is classified according to a second threshold so as to obtain a result of AKI status of the subject.

### RELATED APPLICATIONS

This application claims priority to Taiwan Application Serial Number 109136737, filed Oct. 22, 2020, which is herein incorporated by reference.

### BACKGROUND

### Technical Field

The present disclosure relates to a medical information analysis method and a system thereof. Particularly, the present disclosure relates to a method for assessing acute kidney injury and an acute kidney injury assessment system.

### Description of Related Art

Acute kidney injury (“AKI” hereafter) is a deterioration of renal function within a short period of time which happens suddenly and can be reversible. The patient with AKI will have symptoms of abnormally increasing serum creatinine concentration and urea nitrogen. Furthermore, symptoms such as general fatigue, decreased urine output, edema, loss of appetite, nausea, vomiting, and unconsciousness may happen.

In the aspect of diagnosis, because the AKI does not have obvious indicative clinical symptoms, the main method of clinical diagnosis of the AKI is to test the changes in serum creatinine concentration by a blood draw. However, the basic condition of renal function of the patient is not always available at the first time in an outpatient clinic or hospitalization, so that not only the condition of the patient cannot be known immediately, but also the corresponding treatment will be delayed, resulting in long-term damage to the renal function. Furthermore, the current healthcare institutions do not have a medical system that can comprehensively monitor the changes in renal function of patients admitted in outpatient clinics and emergency department, which has a lot of influence on the selection and implementation of appropriate medical plans.

Therefore, how to develop a method for assessing AKI, which is automated, standardized, rapid and with high detection accuracy, is a technical issue with clinical application value.

### SUMMARY

According to one aspect of the present disclosure, a method for assessing acute kidney injury includes following steps. An acute kidney injury assessing date of a subject is provided. A testing kidney function diagnostic dataset is provided, wherein the testing kidney function diagnostic dataset includes a plurality of serum creatinine concentration data and a plurality of glomerular filtration rate data, and a recording date of each of the serum creatinine concentration data and a recording date of each of the glomerular filtration rate data is on 0 to 180 days before the acute kidney injury assessing date. A preprocessing step is performed, wherein a changing degree over a time period of the serum creatinine concentration data and a changing degree over a time period of the glomerular filtration rate data are respectively calculated so as to obtain a fluctuation value of serum creatinine concentration and a fluctuation value of eGFR. A first classifying step is performed, wherein the fluctuation value of serum creatinine concentration is classified by a calculating classifier according to a first threshold or the fluctuation value of eGFR is classified by the calculating classifier according to a second threshold so as to obtain a result of AKI status of the subject. When the fluctuation value of serum creatinine concentration is larger than the first threshold or the fluctuation value of eGFR is larger than the second threshold, the subject is classified as a patient with AKI. When the fluctuation value of serum creatinine concentration is smaller than the first threshold or the fluctuation value of eGFR is smaller than the second threshold, the subject is classified as a subject without AKI.

According to another aspect of the present disclosure, an acute kidney injury assessment system includes a capturing device and a processor. The capturing device is for capturing a testing kidney function diagnostic dataset of a subject, wherein the testing kidney function diagnostic dataset includes a plurality of serum creatinine concentration data and a plurality of glomerular filtration rate data, and the capturing device automatically captures the serum creatinine concentration data and the glomerular filtration rate data recorded on 0 to 180 days before an acute kidney injury assessing date. The processor is electronically connected to the capturing device, wherein the processor includes an AKI assessing program, and the AKI assessing program is for assessing a status of AKI of the subject when the AKI assessing program is executed by the processor. The AKI assessing program includes a preprocessing model and a first classifying model. The preprocessing model is for respectively calculating a changing degree over a time period of the serum creatinine concentration data and a changing degree over a time period of the glomerular filtration rate data so as to obtain a fluctuation value of serum creatinine concentration and a fluctuation value of eGFR. The first classifying model is for classifying the fluctuation value of serum creatinine concentration by a calculating classifier according to a first threshold or for classifying the fluctuation value of eGFR by the calculating classifier according to a second threshold so as to obtain a result of AKI status of the subject.

### BRIEF DESCRIPTION OF THE DRAWINGS

The present disclosure can be more fully understood by reading the following detailed description of the embodiment, with reference made to the accompanying drawings as follows:

FIG. 1 is a flow chart of a method for assessing acute kidney injury according to the first embodiment of the present disclosure.

FIG. 2 is a flow chart of a method for assessing acute kidney injury according to the second embodiment of the present disclosure.

FIG. 3 is a flow chart of a method for assessing acute kidney injury according to the third embodiment of the present disclosure.

FIG. 4 is a flow chart of a method for assessing acute kidney injury according to the fourth embodiment of the present disclosure.

FIG. 5 is a block diagram of an acute kidney injury assessment system according to the fifth embodiment of the present disclosure.

FIG. 6 is a block diagram of an acute kidney injury assessment system according to the sixth embodiment of the present disclosure.

FIG. 7 is a block diagram of an acute kidney injury assessment system according to the seventh embodiment of the present disclosure.

FIG. 8 is a block diagram of an acute kidney injury assessment system according to the eighth embodiment of the present disclosure.

FIG. 9 is an assessing flow chart of an acute kidney injury assessment system of the present disclosure.

### DETAILED DESCRIPTION

The present disclosure will be further exemplified by the following specific embodiments to facilitate utilizing and practicing the present disclosure completely by the people skilled in the art without over-interpreting and over-experimenting. However, these practical details are used to describe how to implement the materials and methods of the present disclosure and are not necessary.

<The Method for Assessing Acute Kidney Injury of the Present Disclosure>

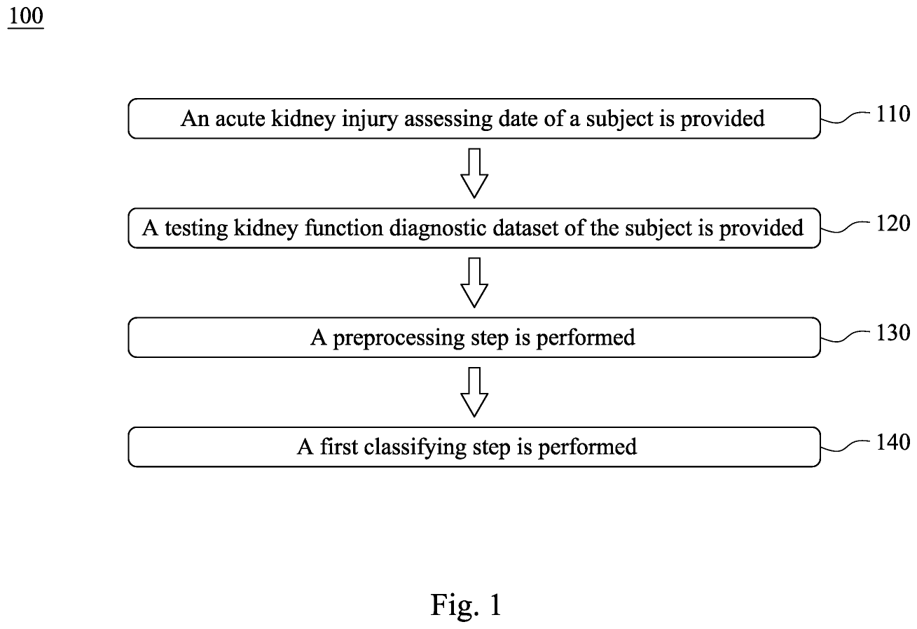

Please refer to FIG. 1, which is a flow chart of a method 100 for assessing acute kidney injury according to the first embodiment of the present disclosure. The method 100 for assessing acute kidney injury 100 includes Step 110, Step 120, Step 130 and Step 140.

In Step 110, an acute kidney injury assessing date of a subject is provided. In detail, the acute kidney injury assessing date can be the date when the subject goes to the hospital or clinic for outpatient or emergency treatment, or the date in which the renal function of the subject is assessed, so as to process the following assessment.

In Step 120, a testing kidney function diagnostic dataset of the subject is provided, wherein the testing kidney function diagnostic dataset includes a plurality of serum creatinine concentration data and a plurality of glomerular filtration rate data, and a recording date of each of the serum creatinine concentration data and a recording date of each of the glomerular filtration rate data is on 0 to 180 days before the acute kidney injury assessing date. In detail, the testing kidney function diagnostic dataset includes all the serum creatinine concentration data recorded on 0 to 180 days before the acute kidney injury assessing date, and the glomerular filtration rate data are calculated based on the serum creatinine concentration data, the age, the gender and the race of the subject according to the Diet in Renal Disease (MDRD) study equation (glomerular filtration rate (eGFR)=186×(Serum creatinine level)^{-1.154}×(age)^{-0.203}×1.212 [if the subject is female, the result must be further×0.742]). The selection of time range within 0 to 180 days before the acute kidney injury assessing date is based on the analysis of current clinical data, and the aforementioned time range represents the longest possible action time that the kidney of older subjects or subjects with chronic kidney disease may be damaged after the administration of nephrotoxic drugs. In other words, the serum creatinine concentration data and the glomerular filtration rate data obtained within the aforementioned time period may be affected by the nephrotoxic drugs, and the subject may be suffered from AKI. Furthermore, the glomerular filtration rate data also can be calculated by other equation except the MDRD study equation, and the present disclosure is not limited thereto.

In Step 130, a preprocessing step is performed, wherein a changing degree over a time period of the serum creatinine concentration data and a changing degree over a time period of the glomerular filtration rate data are respectively calculated so as to obtain a fluctuation value of serum creatinine concentration and a fluctuation value of eGFR. In specific, the serum creatinine concentration data can include a maximum serum creatinine concentration data and a minimum serum creatinine concentration data, and the glomerular filtration rate data can include a maximum glomerular filtration rate data and a minimum glomerular filtration rate data. The fluctuation value of serum creatinine concentration is calculated based on the maximum serum creatinine concentration data and the minimum serum creatinine concentration data according to the fluctuation value of serum creatinine concentration calculating formula (I), and the fluctuation value of eGFR is calculated based on the maximum glomerular filtration rate data and the minimum glomerular filtration rate data according to the fluctuation value of eGFR calculating formula (II). The fluctuation value of serum creatinine concentration calculating formula (I) is shown as follows:

$$\begin{matrix}Fluctuation value of serum \\ creatinine concentration ( \% )\end{matrix} = ( \frac{\vert \begin{matrix}{SCr}_{max} - \\ {SCr}_{min}\end{matrix} \vert}{{SCr}_{min}} ) \times 100 \% ; \qquad ( Formula I )$$

wherein, SCrₘₐₓ represents the value of the maximum serum creatinine concentration data, and SCrₘᵢₙ represents the value of the minimum serum creatinine concentration data. The fluctuation value of eGFR calculating formula (II) is shown as follows:

$$\begin{matrix}Fluctuation value \\ of eGFR ( \% )\end{matrix} = ( \frac{\vert {GFr}_{max} - {GFr}_{min} \vert}{{GFr}_{max}} ) \times 100 \% ; \qquad ( Formula II )$$

wherein, GFRₘₐₓ represents the value of the maximum glomerular filtration rate data, and the GFRₘᵢₙ represents the value of the minimum glomerular filtration rate data.

In Step 140, a first classifying step is performed, wherein the fluctuation value of serum creatinine concentration is classified by a calculating classifier according to a first threshold or the fluctuation value of eGFR is classified by the calculating classifier according to a second threshold so as to obtain a result of AKI status of the subject. When the fluctuation value of serum creatinine concentration is larger than the first threshold or the fluctuation value of eGFR is larger than the second threshold, the subject is classified as a patient with AKI. When the fluctuation value of serum creatinine concentration is smaller than the first threshold or the fluctuation value of eGFR is smaller than the second threshold, the subject is classified as a subject without AKI. In specific, the first threshold can be 50%, and the second threshold can be 35%. In detail, when the fluctuation value of serum creatinine concentration of the subject is larger than 50% or the fluctuation value of eGFR thereof is larger than 35%, the subject has a greater degree of deterioration in the renal function within 0 to 180 days before the acute kidney injury assessing date. In this time, the subject will be classified as the patient with AKI by the calculating classifier of the present disclosure so as to provide appropriate medical plans timely. On the contrary, when the fluctuation value of serum creatinine concentration of the subject is smaller than 50% or the fluctuation value of eGFR is smaller than 35%, the change of the renal function of the subject on 0 to 180 days before the acute kidney injury assessing date is still within the standard value range. Thus, the subject will be classified as a subject without AKI by the calculating classifier of the present disclosure so as to facilitate the formulation of subsequent medical plans and the implementation of treatment measures. Furthermore, the calculating classifier of the present disclosure can be Cox regression calculating classifier (Cox Proportional Hazard Model).

Furthermore, in the method 100 for assessing acute kidney injury of the present disclosure, the testing kidney function diagnostic dataset can further include a physiological age data and a gender data, and a risk prognosis data of the result of AKI status can be adjusted by the calculating classifier based on the physiological age data and the gender data. Thus, the result of AKI status can be more in line with the actual status of the subject so as to enhance the assessing accuracy of the method 100 for assessing acute kidney injury of the present disclosure. Furthermore, the testing kidney function diagnostic dataset can be stored in a physical storage device or a cloud storage device, but the present disclosure is not limited thereto.

Therefore, by analyzing the fluctuation values of the serum creatinine concentration data and the glomerular filtration rate data recorded on 0 to 180 days before the acute kidney injury assessing date by the calculating classifier, the method 100 for assessing acute kidney injury of the present disclosure can assess the AKI status of the subject. Thus, not only it is favorable for calculating and obtaining the medical condition of the subject in time and facilitating the design of subsequent medical plans, but also the long-term impairment of kidney function caused by delays in treatment can be avoided. Hence, the method 100 for assessing acute kidney injury has excellent clinical application potential.

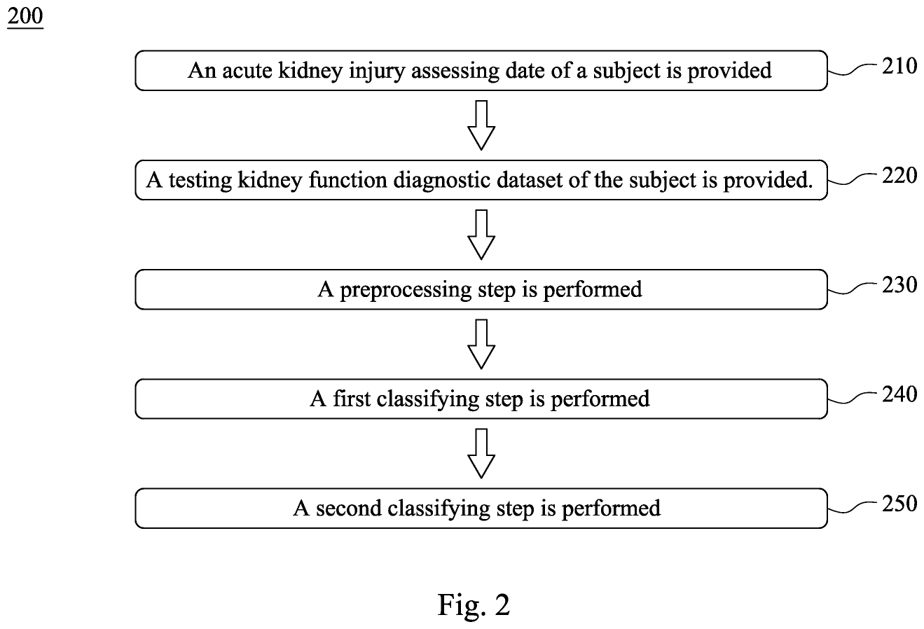

Please refer to FIG. 2, which is a flow chart of a method 200 for assessing acute kidney injury according to the second embodiment of the present disclosure. The method 200 for assessing acute kidney injury includes Step 210, Step 220, Step 230, Step 240 and Step 250, wherein Step 210, Step 220, Step 230 and Step 240 are the same with Step 110, Step 120, Step 130 and Step 140 of FIG. 1, so that the same details there between are not described again herein.

In Step 250, a second classifying step is performed. In detail, the testing kidney function diagnostic dataset of the present disclosure can further include a base serum creatinine concentration data, and the serum creatinine concentration data can include a first serum creatinine concentration data, wherein a recording date of the base serum creatinine concentration data is the acute kidney injury assessing date, and the recording date of the first serum creatinine concentration data is the closest to the acute kidney injury assessing date among all the serum creatinine concentration data. In the second classifying step, the base serum creatinine concentration data and the first serum creatinine concentration data are calculated by the calculating classifier so as to obtain a difference value of serum creatinine concentration, and the difference value of serum creatinine concentration is compared with a threshold of serum creatinine concentration so as to assess a type of AKI of the patient with AKI. In detail, the recording date of the base serum creatinine concentration data is the acute kidney injury assessing date, and the recording date of the first serum creatinine concentration data is the closest to the acute kidney injury assessing date among all of the serum creatinine concentration data. If the base serum creatinine concentration data cannot be obtained in time, the first and the second serum creatinine concentration data are the closest to the acute kidney injury assessing date that are recorded before the acute kidney injury assessing date are captured and the difference there between are calculated so as to obtain a difference value of serum creatinine concentration. When the difference value of serum creatinine concentration is larger than the threshold of serum creatinine concentration, the type of AKI is a deteriorating type of AKI, and when the difference value of serum creatinine concentration is smaller than the threshold of serum creatinine concentration, the type of AKI is a stable type of AKI.

In detail, the first serum creatinine concentration data can be the closest serum creatinine concentration data (that is, the last trackbacking serum creatinine concentration data) obtained before the acute kidney injury assessing date, the serum creatinine concentration data obtained on the acute kidney injury assessing date is the base serum creatinine concentration data, and the recording date of the base serum creatinine concentration data must be later than the recording date of the first serum creatinine concentration data. That is, the second classifying step can further analyze the difference of two of the serum creatinine concentration data recorded before the acute kidney injury assessing date.

The difference value of serum creatinine concentration is calculated according to the difference value of serum creatinine concentration calculating formula (III), and the difference value of serum creatinine concentration calculating formula (III) is shown as follows:

$$Difference value of serum creatinine concentration = SC {r}_{L a s t} - S C {r}_{L ast - 1} ; \qquad ( Formula III )$$

wherein, SCr_{Last} represents the value of the base serum creatinine concentration data, and SCr_{Last-1} represents the value of the first serum creatinine concentration data. In specific, the threshold of serum creatinine concentration can be 0.3 mg/dL. If the difference value of serum creatinine concentration is larger than 0.3 mg/dL, it represents that the serum creatinine concentration of the patient with AKI has a larger increase and deterioration before the acute kidney injury assessing date, and the medical status of the patient with AKI is rapidly deteriorating. Accordingly, the type of AKI of the patient with AKI is classified as a deteriorating type of AKI by the calculating classifier of the present disclosure. On the contrary, if the difference value of serum creatinine concentration is smaller than 0.3 mg/dL, the medical status of the patient with AKI is without rapidly deteriorating, so that the type of AKI of the patient with AKI is classified as a stable type of AKI by the calculating classifier of the present disclosure.

Therefore, by analyzing the fluctuation values of the serum creatinine concentration data and the glomerular filtration rate data recorded on 0 to 180 days before the acute kidney injury assessing date by the calculating classifier, the method 200 for assessing acute kidney injury of the present disclosure can assess the AKI status of the subject, and the type of AKI of the patient with AKI can be further analyzed by the calculating classifier so as to facilitate the design of subsequent medical plans. Hence, the method 200 for assessing acute kidney injury has excellent clinical application potential.

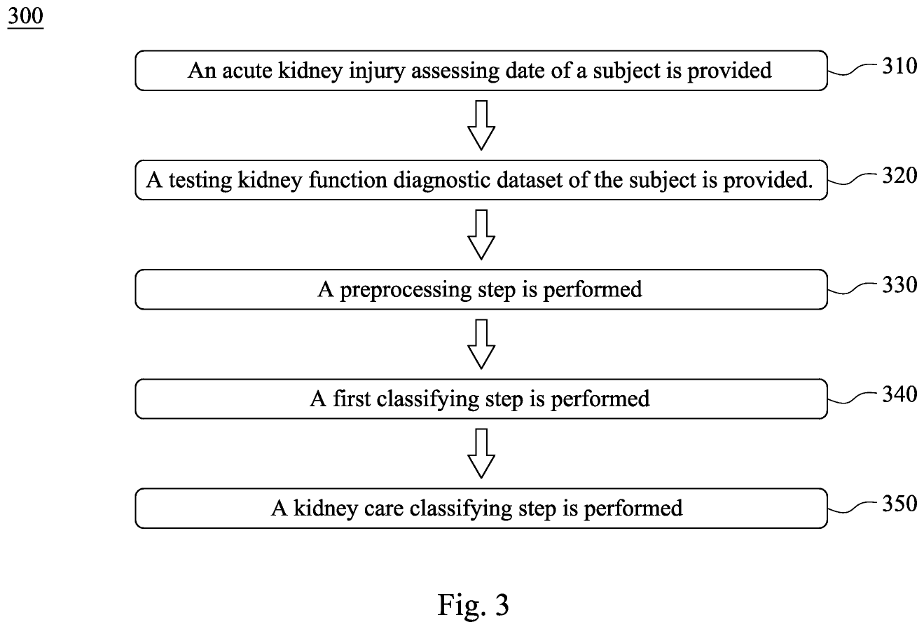

Please refer to FIG. 1, FIG. 2 and FIG. 3 simultaneously, wherein FIG. 3 is a flow chart of a method 300 for assessing acute kidney injury according to the third embodiment of the present disclosure. The method 300 for assessing acute kidney injury includes Step 310, Step 320, Step 330, Step 340 and Step 350, wherein Step 310, Step 320, Step 330 and Step 340 are the same with Step 110, Step 120, Step 130 and Step 140 of FIG. 1, so that the same details there between are not described again herein.

In Step 350, a kidney care classifying step is performed. In detail, the testing kidney function diagnostic dataset can further include a base glomerular filtration rate data, and a recording date of the base glomerular filtration rate data is the acute kidney injury assessing date. In the kidney care classifying step, the base glomerular filtration rate data is analyzed by the calculating classifier of the present disclosure so as to obtain a result of kidney care classification. In detail, the method 300 for assessing acute kidney injury can be used with the results of the method 100 for assessing acute kidney injury and the method 200 for assessing acute kidney injury so as to respectively analyze the values of the base glomerular filtration rate data of the subject without AKI, the patient with the deteriorating type of AKI and the patient with the stable type of AKI, and then output the corresponding results of kidney care classification. Furthermore, the doctors of outpatient clinics and emergency department can design appropriate medical plans according to the aforementioned results of kidney care classification so as to prevent the long-term damage to the renal function caused by delays in treatment. Please refer to Table 1, which shows the values of base glomerular filtration rate data and the corresponding results of kidney care classification thereof, so that it is favorable for outputting a correct result of kidney care classification under the premise that the current clinical diagnostic standards are satisfied.

TABLE 1

| | Subject | | |
|---|---|---|---|
| eGFR | | Stable | Deteriorating |
| (ml/min/1.73 m²) | Without AKI | type of AKI | type of AKI |
| eGFR < 45 | Referral to | Referral to | Mandatory referral |
| | nephrology | nephrology | to nephrology |
| 45 ≤ eGFR < 60 | Track renal | Track renal | Track renal |
| | function | function or | function or |
| | | referral to | referral to |
| | | nephrology | nephrology |
| 60 ≤ eGFR | Normal renal | Track renal | Track renal |
| | function | function | function |

Therefore, by analyzing the last glomerular filtration rate data recorded before the acute kidney injury assessing date or analyzing the base glomerular filtration rate data recorded on the acute kidney injury assessing date by the calculating classifier, the method 300 for assessing acute kidney injury can output a corresponding result of kidney care classification. Hence, it is favorable for designing the subsequent medical plans by the doctor, and thus the method 300 for assessing acute kidney injury has excellent clinical application potential.

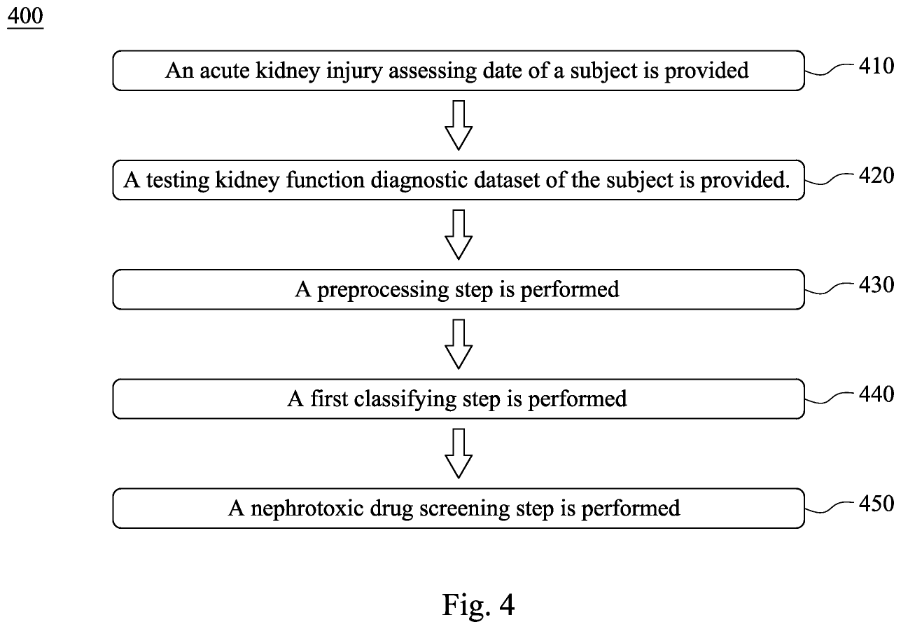

Please refer to FIG. 4 is a flow chart of a method 400 for assessing acute kidney injury according to the fourth embodiment of the present disclosure. The method 400 for assessing acute kidney injury includes Step 410, Step 420, Step 430, Step 440 and Step 450, wherein Step 410, Step 420, Step 430 and Step 440 are the same with Step 110, Step 120, Step 130 and Step 140 of FIG. 1, so that the same details there between are not described again herein.

In Step 450, a nephrotoxic drug screening step is performed, wherein the testing kidney function diagnostic dataset can further include a drug utilization data, and the drug utilization data is analyzed in the nephrotoxic drug screening step so as to output a screening result of nephrotoxic drug usage. In detail, the nephrotoxic drug includes six kinds of drugs including non-steroidal anti-inflammatory drugs (NSAIDs), radiocontrast, antimicrobials, chemotherapy and immunotherapy, renin-angiotension system blocker (ARB/ACEi) and diuretics which can affect the renal function of a subject, so that it is favorable for analyzing and assessing whether the cause of AKI of the subject is due to the influences of nephrotoxic drugs. Accordingly, the method 400 for assessing acute kidney injury of the present disclosure has excellent clinical application potential.

<The Acute Kidney Injury Assessment System of the Present Disclosure>

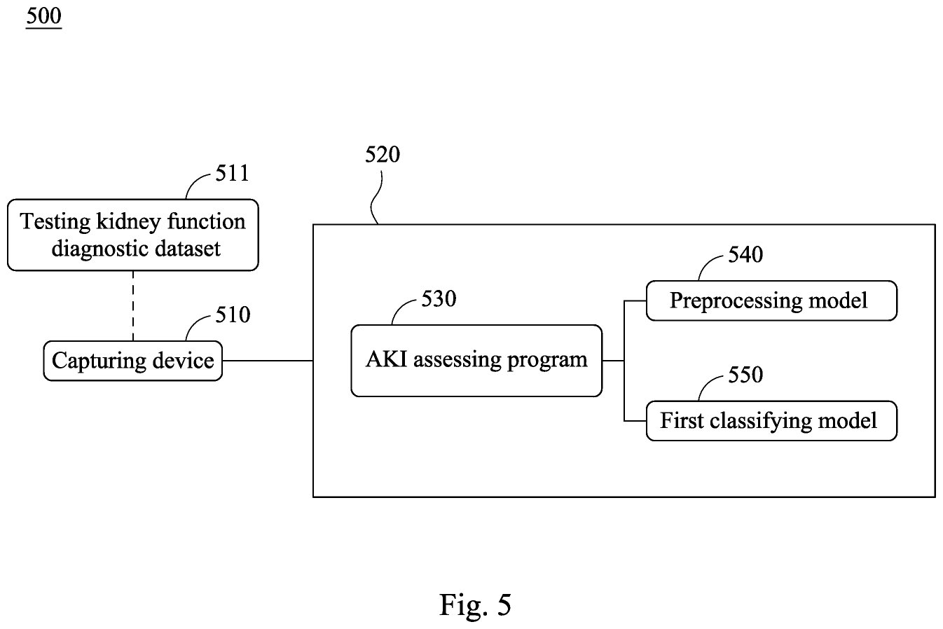

Please refer to FIG. 5, which is a block diagram of an acute kidney injury assessment system 500 according to the fifth embodiment of the present disclosure. The acute kidney injury assessment system 500 includes a capturing device 510 and a processor 520.

The capturing device 510 is for capturing a testing kidney function diagnostic dataset 511 of a subject, wherein the testing kidney function diagnostic dataset 511 includes a plurality of serum creatinine concentration data and a plurality of glomerular filtration rate data, and the capturing device 510 automatically captures the serum creatinine concentration data and the glomerular filtration rate data recorded on 0 to 180 days before an acute kidney injury assessing date. In detail, the acute kidney injury assessing date can be the date when the subject goes to the hospital or clinic for outpatient or emergency treatment, or the date in which the renal function of the subject will be assessed for the following assessment. Furthermore, the testing kidney function diagnostic dataset 511 includes all the serum creatinine concentration data recorded on 0 to 180 days before the acute kidney injury assessing date and the glomerular filtration rate data are calculated based on the serum creatinine concentration data. According to the analysis of current clinical data, the time range that the kidney of older subjects or subjects with chronic kidney disease may be damaged is 0 to 180 days after the potential exposure to nephrotoxic drugs, and the serum creatinine concentration data and the glomerular filtration rate data obtained within the aforementioned time period may be affected by the nephrotoxic drugs, so that the subject may be suffered from AKI. Accordingly, the acute kidney injury assessment system 500 is used to analyze the serum creatinine concentration data and the glomerular filtration rate data recorded on 0 to 180 days before the acute kidney injury assessing date. Furthermore, the testing kidney function diagnostic dataset 511 can be stored in a physical storage device or a cloud storage device, but the present disclosure is not limited thereto.

The processor 520 is electronically connected to the capturing device 510, wherein the processor 520 includes an AKI assessing program 530. The AKI assessing program 530 is for assessing a status of AKI of the subject when the AKI assessing program 530 is executed by the processor 520. The AKI assessing program 530 includes a preprocessing model 540 and a first classifying model 550.

The preprocessing model 540 is for respectively calculating a changing degree over a time period of the serum creatinine concentration data and a changing degree over a time period of the glomerular filtration rate data so as to obtain a fluctuation value of serum creatinine concentration and a fluctuation value of eGFR. The serum creatinine concentration data can include a maximum serum creatinine concentration data and a minimum serum creatinine concentration data, and the glomerular filtration rate data can include a maximum glomerular filtration rate data and a minimum glomerular filtration rate data. The fluctuation value of serum creatinine concentration is calculated based on the maximum serum creatinine concentration data and the minimum serum creatinine concentration data according to the aforementioned fluctuation value of serum creatinine concentration calculating formula (I), and the fluctuation value of eGFR is calculated based on the maximum glomerular filtration rate data and the minimum glomerular filtration rate data according to the aforementioned fluctuation value of eGFR calculating formula (II). The details of the fluctuation value of serum creatinine concentration calculating formula (I) and the fluctuation value of eGFR calculating formula (II) are shown in the aforementioned description, and they are not described again herein.

The first classifying model 550 is for classifying the fluctuation value of serum creatinine concentration by a calculating classifier according to a first threshold or for classifying the fluctuation value of eGFR by the calculating classifier according to a second threshold so as to obtain a result of AKI status of the subject. When the fluctuation value of serum creatinine concentration is larger than the first threshold or the fluctuation value of eGFR is larger than the second threshold, the subject is classified as a patient with AKI. When the fluctuation value of serum creatinine concentration is smaller than the first threshold or the fluctuation value of eGFR is smaller than the second threshold, the subject is classified as a subject without AKI. In specific, the first threshold can be 50%, and the second threshold can be 35%. Thus, it is favorable for assessing that whether the subject has a greater degree of deterioration in the renal function within 0 to 180 days before the acute kidney injury assessing date so as to facilitate the formulation of subsequent medical plans and the implementation of treatment measures. Furthermore, the calculating classifier of the present disclosure can be Cox regression calculating classifier.

Furthermore, in the acute kidney injury assessment system 500 of the present disclosure, the testing kidney function diagnostic dataset 511 can further include a physiological age data and a gender data, and a risk prognosis data of the result of AKI status can be adjusted by the calculating classifier based on the physiological age data and the gender data. Thus, the result of AKI status can be more in line with the actual status of the subject so as to enhance the assessing accuracy of the acute kidney injury assessment system 500.

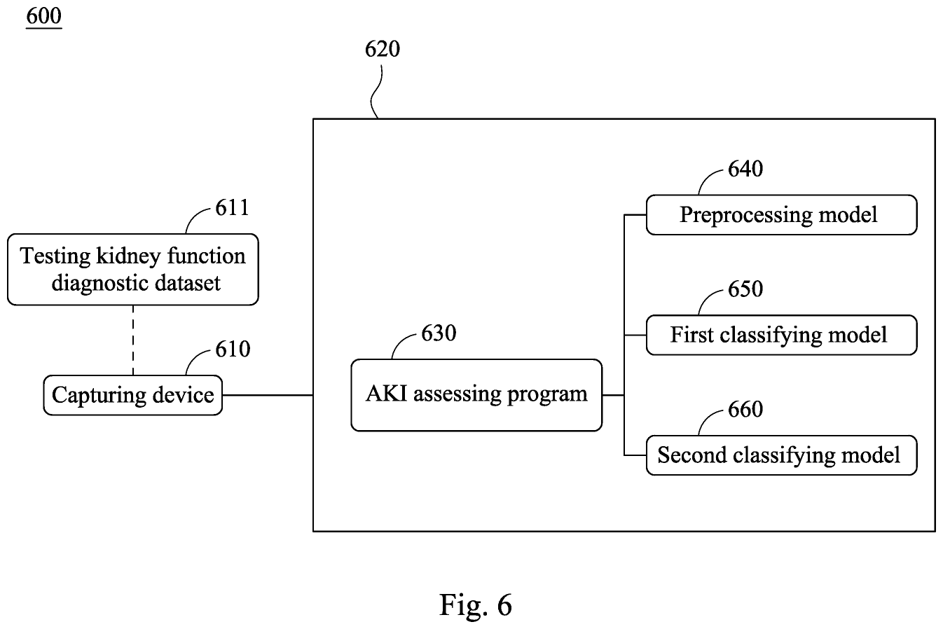

Please refer to FIG. 6, which is a block diagram of an acute kidney injury assessment system 600 according to the sixth embodiment of the present disclosure. The acute kidney injury assessment system 600 includes a capturing device 610 and a processor 620. The capturing device 610 is for capturing a testing kidney function diagnostic dataset 611 of a subject, and the processor 620 includes an AKI assessing program 630, wherein the capturing device 610 and the processor 620 are similar with the capturing device 510 and the processor 520 of FIG. 5, and the same details there between are not described again herein.

The AKI assessing program 630 includes a preprocessing model 640, a first classifying model 650 and a second classifying model 660, wherein the preprocessing model 640 and the first classifying model 650 are the same with the preprocessing model 540 and the first classifying model 550 of FIG. 5, and the same details there between are not described again herein. In detail, the testing kidney function diagnostic dataset 611 can further include a base serum creatinine concentration data, and the serum creatinine concentration data can include a first serum creatinine concentration data, wherein a recording date of the base serum creatinine concentration data is the acute kidney injury assessing date, and the recording date of the first serum creatinine concentration data is the closest to the acute kidney injury assessing date among all the serum creatinine concentration data. The second classifying model 660 is for calculating the base serum creatinine concentration data and the first serum creatinine concentration data by the calculating classifier of the present disclosure so as to obtain a difference value of serum creatinine concentration, and the difference value of serum creatinine concentration is compared with a threshold of serum creatinine concentration so as to assess a type of AKI of the patient with AKI. In detail, the recording date of the base serum creatinine concentration data is the acute kidney injury assessing date, and the recording date of the first serum creatinine concentration data is the closest to the acute kidney injury assessing date among all of the serum creatinine concentration data. If the base serum creatinine concentration data cannot be obtained in time, the first and the second serum creatinine concentration data are the closest to the acute kidney injury assessing date that are recorded before the acute kidney injury assessing date are captured and the difference there between are calculated so as to obtain a difference value of serum creatinine concentration. When the difference value of serum creatinine concentration is larger than the threshold of serum creatinine concentration, the type of AKI is a deteriorating type of AKI. When the difference value of serum creatinine concentration is smaller than the threshold of serum creatinine concentration, the type of AKI is a stable type of AKI.

In detail, the first serum creatinine concentration data can be the closest serum creatinine concentration data (that is, the last trackbacking serum creatinine concentration data) obtained before the acute kidney injury assessing date, the serum creatinine concentration data obtained on the acute kidney injury assessing date is the base serum creatinine concentration data, and the difference value of serum creatinine concentration is calculated according to the difference value of serum creatinine concentration calculating formula (III). The details of the difference value of serum creatinine concentration calculating formula (III) are shown in the aforementioned description, and it is not described again herein.

In specific, the threshold of serum creatinine concentration can be 0.3 mg/dL. If the difference value of serum creatinine concentration is larger than 0.3 mg/dL, it represents that the serum creatinine concentration of the patient with AKI has a larger increase and deterioration before the acute kidney injury assessing date, and the medical status of the patient with AKI is rapidly deteriorating. Accordingly, the type of AKI of the patient with AKI is classified as a deteriorating type of AKI. On the contrary, if the difference value of serum creatinine concentration is smaller than 0.3 mg/dL, the medical status of the patient with AKI is without rapidly deteriorating, so that the type of AKI of the patient with AKI is classified as a stable type of AKI.

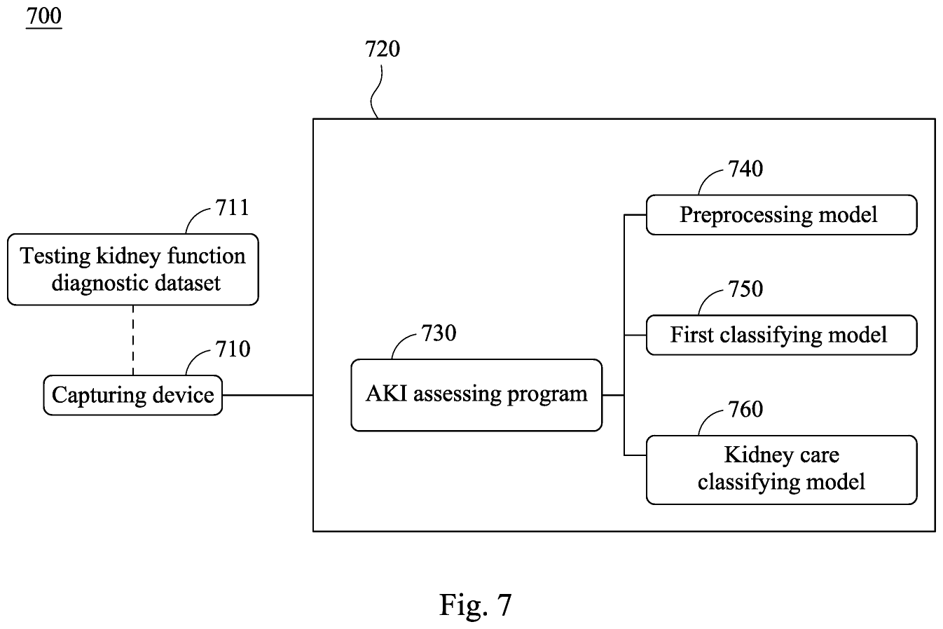

Please refer to FIG. 7, which is a block diagram of an acute kidney injury assessment system 700 according to the seventh embodiment of the present disclosure. The acute kidney injury assessment system 700 includes a capturing device 710 and a processor 720. The capturing device 710 is for capturing a testing kidney function diagnostic dataset 711 of a subject, and the processor 720 includes an AKI assessing program 730, wherein the capturing device 710 and the processor 720 are similar with the capturing device 510 and the processor 520 of FIG. 5, and the same details there between are not described again herein.

The AKI assessing program 730 includes a preprocessing model 740, a first classifying model 750 and a kidney care classifying model 760, wherein the preprocessing model 740 and the first classifying model 750 are the same with the preprocessing model 540 and the first classifying model 550 of FIG. 5, and the same details there between are not described again herein. In detail, the testing kidney function diagnostic dataset 711 can further include a base glomerular filtration rate data, and a recording date of the base glomerular filtration rate data is the acute kidney injury assessing date. The kidney care classifying model 760 is for analyzing the base glomerular filtration rate data by the calculating classifier of the present disclosure so as to obtain a result of kidney care classification.

In detail, the base glomerular filtration rate data for calculating the deteriorating type or the stable type of AKI is recorded the closest to the acute kidney injury assessing date or is recorded on the acute kidney injury assessing date, and the calculating classifier of the present disclosure can respectively analyze the values of the base glomerular filtration rate data of the subject without AKI, the patient with the deteriorating type of AKI and the patient with the stable type of AKI and then output the corresponding results of kidney care classification. Furthermore, the doctors of outpatient clinics and emergency department can design appropriate medical plans according to the aforementioned results of kidney care classification so as to prevent the long-term damage to the renal function caused by delays in treatment. Moreover, the values of base glomerular filtration rate data and the corresponding results of kidney care classification thereof as shown in Table 1, and they are not described again herein.

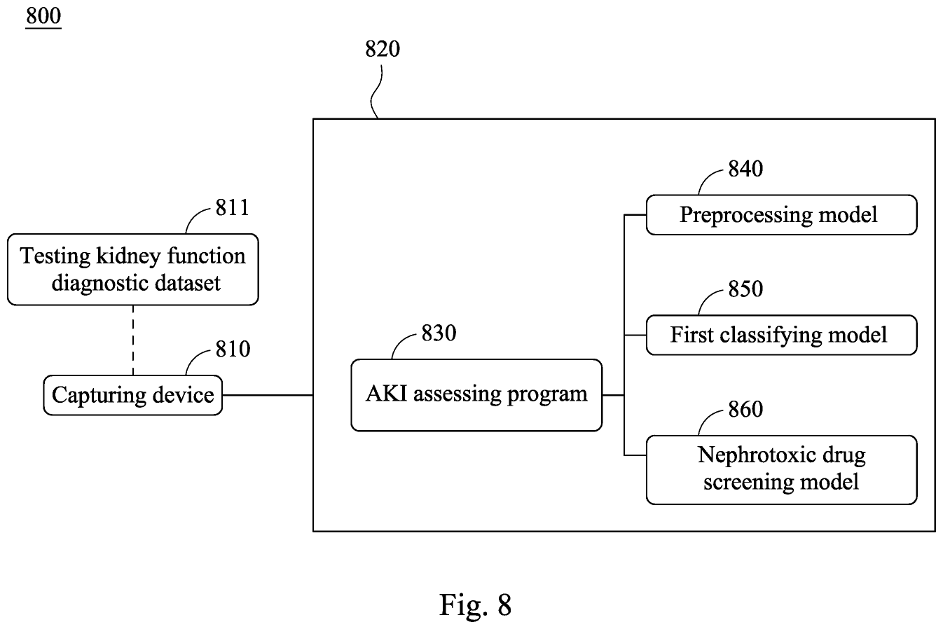

Please refer to FIG. 8, which is a block diagram of an acute kidney injury assessment system 800 according to the eighth embodiment of the present disclosure. The acute kidney injury assessment system 800 includes a capturing device 810 and a processor 820. The capturing device 810 is for capturing a testing kidney function diagnostic dataset 811 of a subject, and the processor 820 includes an AKI assessing program 830, wherein the capturing device 810 and the processor 820 are similar with the capturing device 510 and the processor 520 of FIG. 5, and the same details there between are not described again herein.

The AKI assessing program 830 includes a preprocessing model 840, a first classifying model 850 and a nephrotoxic drug screening model 860, wherein the preprocessing model 840 and the first classifying model 850 are the same with the preprocessing model 540 and the first classifying model 550 of FIG. 5, and the same details there between are not described again herein. In detail, the testing kidney function diagnostic dataset 811 can further include a drug utilization data, and the nephrotoxic drug screening model 860 is for analyzing the drug utilization data so as to output a screening result of nephrotoxic drug usage. Thus, it is favorable for analyzing and assessing whether the cause of AKI of the subject is due to the influences of non-steroidal anti-inflammatory drugs (NSAIDs), radiocontrast, antimicrobials, chemotherapy and immunotherapy, renin-angiotension system blocker (ARB/ACEi), diuretics and other nephrotoxic drugs, so that the acute kidney injury assessment system 800 of the present disclosure has excellent clinical application potential.

Therefore, by analyzing the fluctuation values of the serum creatinine concentration data and the glomerular filtration rate data recorded on 0 to 180 days before the acute kidney injury assessing date by the calculating classifier, the acute kidney injury assessment system 500, the acute kidney injury assessment system 600, the acute kidney injury assessment system 700 and the acute kidney injury assessment system 800 of the present disclosure can assess the acute kidney injury status of the subject. Furthermore, the acute kidney injury assessment system 500, the acute kidney injury assessment system 600, the acute kidney injury assessment system 700 and the acute kidney injury assessment system 800 of the present disclosure can further calculate the prognostic risk instantly and then obtain the possible cause of AKI of the subject by calculating the risk matrix by the calculating classifier and then generalizing the conditions of the comprehensive type of AKI, results of kidney care classification and the nephrotoxic drug usage of the patient with AKI. Hence, it is favorable for designing the subsequent medical plans, and the long-term impairment of kidney function caused by delays in treatment can be avoided, so that the acute kidney injury assessment system 500, the acute kidney injury assessment system 600, the acute kidney injury assessment system 700 and the acute kidney injury assessment system 800 has excellent clinical application potential.

### Example

I. Testing Kidney Function Diagnostic Dataset

The testing kidney function diagnostic dataset used in the present disclosure is a testing kidney function diagnostic dataset collected by China Medical University Hospital including 6,046 patients suffered from the end-stage renal disease (ESRD), and the patients are aged from 20 to 90 years old, wherein the aforementioned ESRD patients have not undergone hemodialysis treatment and have at least two glomerular filtration rate data recorded. The aforementioned data and results are recorded in the electronic medical record so as to facilitate the following analysis. The aforementioned clinical research study is approved by China Medical University & Hospital Research Ethics Committee, which are numbered as CMUH105-REC3-068.

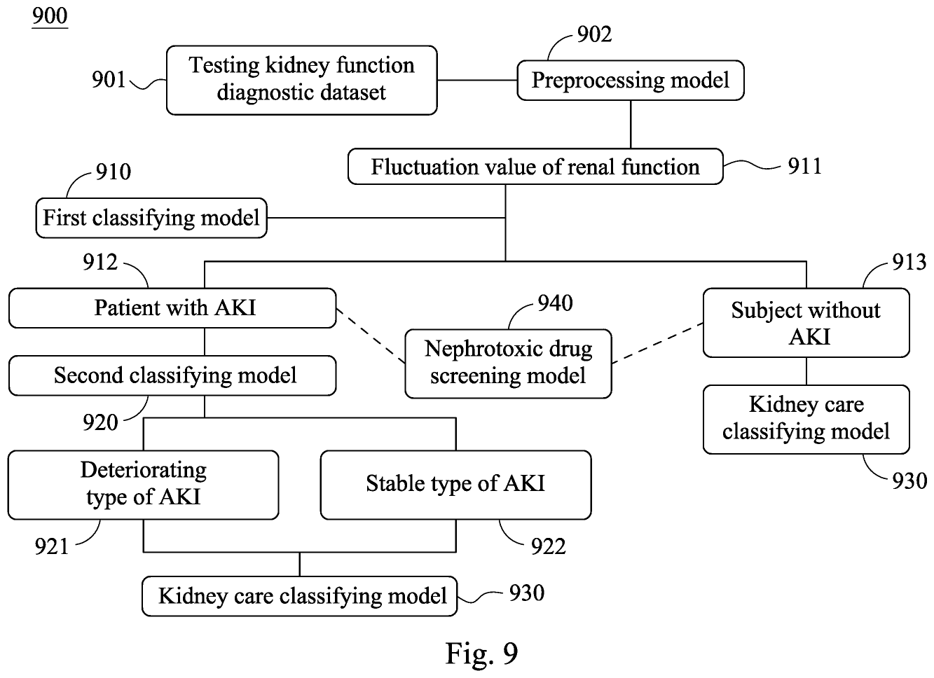

Please refer to FIG. 9, which is an assessing flow chart of an acute kidney injury assessment system 900 of the present disclosure. The clinical application details of the acute kidney injury assessment system 900 and the method for assessing acute kidney injury will be further described according to FIG. 9.

As shown in FIG. 9, the testing kidney function diagnostic dataset 901 stored in a physical storage device or a cloud storage device and including the data of 6,046 patients are respectively analyzed by the first classifying model 910. In detail, the testing kidney function diagnostic dataset 901 includes all of serum creatinine concentration data recorded on 0 to 180 days before the acute kidney injury assessing date and the glomerular filtration rate data calculated based on the serum creatinine concentration data. The aforementioned serum creatinine concentration data and the aforementioned glomerular filtration rate data will be calculated in advance by the preprocessing model 902 so as to obtain a fluctuation value of renal function 911 which changes over time, wherein the fluctuation value of renal function 911 includes a fluctuation value of serum creatinine concentration and a fluctuation value of eGFR.

Next, the first classifying model 910 will classify the fluctuation value of serum creatinine concentration according to a first threshold 50% or classify the fluctuation value of eGFR according to a second threshold 35% by a calculating classifier so as to obtain a result of AKI status of the subject. When the fluctuation value of serum creatinine concentration is larger than 50% or the fluctuation value of eGFR is larger than 35%, the subject is classified as a patient with AKI 912. When the fluctuation value of serum creatinine concentration is smaller than 50% or the fluctuation value of eGFR is smaller than 35%, the subject is classified as a subject without AKI 913.

Then, the patient with AKI 912 and the subject without AKI 913 will be further analyzed, respectively. In the analysis of the patient with AKI 912, the base serum creatinine concentration data and the serum creatinine concentration data of the patient with AKI 912 will be calculated by the calculating classifier of the present disclosure in the second classifying model 920 so as to obtain a difference value of serum creatinine concentration. The difference value of serum creatinine concentration will be compared with a threshold of serum creatinine concentration being 0.3 mg/dL so as to assess a type of AKI of the patient with AKI 912. When the difference value of serum creatinine concentration is larger than 0.3 mg/dL, the type of AKI belongs to a deteriorating type of AKI 921, and when the difference value of serum creatinine concentration is smaller than 0.3 mg/dL, the type of AKI belongs to a stable type of AKI 922.

Furthermore, a kidney care classification of the subject without AKI 913, the patients with AKI 912 with the deteriorating type of AKI 921 and with the stable type of AKI 922 can be assessed by the kidney care classifying model 930 of the acute kidney injury assessment system 900 of the present disclosure. In detail, the testing kidney function diagnostic dataset 901 of each of the subject without AKI 913, the patients with AKI 912 with the deteriorating type of AKI 921 and with the stable type of AKI 922 includes a base glomerular filtration rate data, and the recording date of the base glomerular filtration rate data is the closest date backtracking from the acute kidney injury assessing date. Then, the base glomerular filtration rate data is analyzed by the calculating classifier of the present disclosure in the kidney care classifying model 930 so as to obtain a result of kidney care classification. Thus, it is favorable for the doctors of outpatient clinics and emergency department to design appropriate and different medical plans for the subject without AKI 913, the patients with AKI 912 with the deteriorating type of AKI 921 and with the stable type of AKI 922 according to the aforementioned results of kidney care classification so as to prevent the long-term damage to the renal function caused by delays in treatment.

In addition, the drug utilization data of both of the subject without AKI 913 and the patient with AKI 912 can be further assessed by the nephrotoxic drug screening model 940 so as to analyze and assess whether the cause of AKI of the subject is due to the influences of non-steroidal anti-inflammatory drugs (NSAIDs), radiocontrast, antimicrobials, chemotherapy and immunotherapy, renin-angiotension system blocker (ARB/ACEi), diuretics and other nephrotoxic drugs. The nephrotoxic drug screening model 940 is set up by the list of nephrotoxic drugs confirmed by nephrologists first, and then the aforementioned nephrotoxic drugs will be further selected based on the anatomical therapeutic chemical classification system (ATC code) of World Health Organization as well as the drug list and the drug code published by National Health Insurance Agency. In the aspect of the compounding drugs, the compounding drug will be separated based on the pharmacological ingredients and then further classified so as to enhance the screening efficiency thereof.

Furthermore, the incidence of AKI in the outpatient clinics and emergency department is about 11.9%, and the acute kidney injury assessment system 900 of the present disclosure can analyze the changing degree of the serum creatinine concentration data and the glomerular filtration rate data recorded on 0 to 180 days before the acute kidney injury assessing date by the calculating classifier of the present disclosure so as to immediately and quickly assess the AKI status of the subject and then increase the diagnostic efficiency of AKI.

Moreover, the method for assessing acute kidney injury and the acute kidney injury assessment system of the present disclosure are the first invention disclosed in the field for detecting the AKI in the outpatient clinics and emergency department so as to obtain the status of AKI of the patients admitted in emergency department. Before the method for assessing acute kidney injury and the acute kidney injury assessment system of the present disclosure are disclosed, the diagnosis rate of AKI in outpatient clinics is very low. In average, the method for assessing acute kidney injury and the acute kidney injury assessment system of the present disclosure can diagnose more than 85% of the patients suffered from AKI compared to the conventional method. Furthermore, the diagnosis of AKI is simply based on the clinical data, which is similar with the diagnosis of chronic kidney injury, and when the eGFR of one patient is lower than 60 ml/min/1.73 m², the patient can be diagnosed as a patient with AKI. Therefore, the analyzing accuracy of the method for assessing acute kidney injury and the acute kidney injury assessment system of the present disclosure is affected by the numbers of measure of the serum creatinine concentration when the patient is in the outpatient treatments. Thus, the numbers of measure of the serum creatinine concentration thereof should be larger than two so as to facilitate the assessment of the method for assessing acute kidney injury and the acute kidney injury assessment system of the present disclosure.

Therefore, by analyzing the changing degree of the serum creatinine concentration data and the glomerular filtration rate data recorded on 0 to 180 days before the acute kidney injury assessing date by the calculating classifier, the method for assessing acute kidney injury and the acute kidney injury assessment system of the present disclosure can assess the acute kidney injury status of the subject. Thus, not only it is favorable for obtaining the prognostic risk of the subject timely so as to facilitate the design of subsequent medical plans, but also the long-term impairment of kidney function caused by delays in treatment can be avoided. Hence, the method for assessing acute kidney injury and the acute kidney injury assessment system of the present disclosure have excellent clinical application potential.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure covers modifications and variations of this disclosure provided they fall within the scope of the following claims.

What is claimed is:

1. A method for assessing acute kidney injury, comprising:
providing an acute kidney injury assessing date of a subject;
providing a testing kidney function diagnostic dataset, wherein the testing kidney function diagnostic dataset comprises a plurality of serum creatinine concentration data and a plurality of glomerular filtration rate data, and a recording date of each of the serum creatinine concentration data and a recording date of each of the glomerular filtration rate data is on 0 to 180 days before the acute kidney injury assessing date;
performing a preprocessing step, wherein a changing degree over a time period of the serum creatinine concentration data and a changing degree over a time period of the glomerular filtration rate data are respectively calculated so as to obtain a fluctuation value of serum creatinine concentration and a fluctuation value of eGFR; and
performing a first classifying step, wherein the fluctuation value of serum creatinine concentration is classified by a calculating classifier according to a first threshold or the fluctuation value of eGFR is classified by the calculating classifier according to a second threshold so as to obtain a result of AKI status of the subject;
wherein when the fluctuation value of serum creatinine concentration is larger than the first threshold or the fluctuation value of eGFR is larger than the second threshold, the subject is classified as a patient with AKI;
wherein when the fluctuation value of serum creatinine concentration is smaller than the first threshold or the fluctuation value of eGFR is smaller than the second threshold, the subject is classified as a subject without AKI.

2. The method for assessing acute kidney injury of claim 1, wherein the serum creatinine concentration data comprise a maximum serum creatinine concentration data and a minimum serum creatinine concentration data, the glomerular filtration rate data comprise a maximum glomerular filtration rate data and a minimum glomerular filtration rate data, the fluctuation value of serum creatinine concentration is calculated based on the maximum serum creatinine concentration data and the minimum serum creatinine concentration data, and the fluctuation value of eGFR is calculated based on the maximum glomerular filtration rate data and the minimum glomerular filtration rate data.

3. The method for assessing acute kidney injury of claim 1, wherein the first threshold is 50%, and the second threshold is 35%.

4. The method for assessing acute kidney injury of claim 1, wherein the calculating classifier is Cox regression calculating classifier.

5. The method for assessing acute kidney injury of claim 1, wherein the testing kidney function diagnostic dataset further comprises a base serum creatinine concentration data, the serum creatinine concentration data comprise a first serum creatinine concentration data, a recording date of the base serum creatinine concentration data is the acute kidney injury assessing date, and a recording date of the first serum creatinine concentration data is the closest to the acute kidney injury assessing date among all the serum creatinine concentration data;
wherein the method for assessing acute kidney injury further comprises:
performing a second classifying step, wherein the base serum creatinine concentration data and the first serum creatinine concentration data are calculated by the calculating classifier so as to obtain a difference value of serum creatinine concentration, and the difference value of serum creatinine concentration is compared with a threshold of serum creatinine concentration so as to assess a type of AKI of the patient with AKI;
wherein when the difference value of serum creatinine concentration is larger than the threshold of serum creatinine concentration, the type of AKI is a deteriorating type of AKI;
wherein when the difference value of serum creatinine concentration is smaller than the threshold of serum creatinine concentration, the type of AKI is a stable type of AKI.

6. The method for assessing acute kidney injury of claim 5, wherein the threshold of serum creatinine concentration is 0.3 mg/dL.

7. The method for assessing acute kidney injury of claim 1, wherein the testing kidney function diagnostic dataset further comprises a physiological age data and a gender data, and a risk prognosis data of the result of AKI status is adjusted by the calculating classifier based on the physiological age data and the gender data.

8. The method for assessing acute kidney injury of claim 1, wherein the testing kidney function diagnostic dataset further comprises a base glomerular filtration rate data, and a recording date of the base glomerular filtration rate data is the acute kidney injury assessing date;
wherein the method for assessing acute kidney injury further comprises:
performing a kidney care classifying step, wherein the base glomerular filtration rate data is analyzed by the calculating classifier so as to obtain a result of kidney care classification.

9. The method for assessing acute kidney injury of claim 1, further comprising:
performing a nephrotoxic drug screening step, wherein the testing kidney function diagnostic dataset further comprises a drug utilization data, and the drug utilization data is analyzed in the nephrotoxic drug screening step so as to output a screening result of nephrotoxic drug usage.

10. An acute kidney injury assessment system, comprising:
a capturing device for capturing a testing kidney function diagnostic dataset of a subject, wherein the testing kidney function diagnostic dataset comprises a plurality of serum creatinine concentration data and a plurality of glomerular filtration rate data, and the capturing device automatically captures the serum creatinine concentration data and the glomerular filtration rate data recorded on 0 to 180 days before an acute kidney injury assessing date; and
a processor electronically connected to the capturing device, wherein the processor comprises an AKI assessing program, and the AKI assessing program is for assessing a status of acute kidney injury of the subject when the AKI assessing program is executed by the processor;
wherein the AKI assessing program comprises:
a preprocessing model for respectively calculating a changing degree over a time period of the serum creatinine concentration data and a changing degree over a time period of the glomerular filtration rate data so as to obtain a fluctuation value of serum creatinine concentration and a fluctuation value of eGFR; and
a first classifying model for classifying the fluctuation value of serum creatinine concentration by a calculating classifier according to a first threshold or for classifying the fluctuation value of eGFR by the calculating classifier according to a second threshold so as to obtain a result of AKI status of the subject;
wherein when the fluctuation value of serum creatinine concentration is larger than the first threshold or the fluctuation value of eGFR is larger than the second threshold, the subject is classified as a patient with AKI;
wherein when the fluctuation value of serum creatinine concentration is smaller than the first threshold or the fluctuation value of eGFR is smaller than the second threshold, the subject is classified as a subject without AKI.

11. The acute kidney injury assessment system of claim 10, wherein the serum creatinine concentration data comprise a maximum serum creatinine concentration data and a minimum serum creatinine concentration data, the glomerular filtration rate data comprise a maximum glomerular filtration rate data and a minimum glomerular filtration rate data, the fluctuation value of serum creatinine concentration is calculated based on the maximum serum creatinine concentration data and the minimum serum creatinine concentration data, and the fluctuation value of eGFR is calculated based on the maximum glomerular filtration rate data and the minimum glomerular filtration rate data.

12. The acute kidney injury assessment system of claim 10, wherein the first threshold is 50%, and the second threshold is 35%.

13. The acute kidney injury assessment system of claim 10, wherein the calculating classifier is Cox regression calculating classifier.

14. The acute kidney injury assessment system of claim 10, wherein the testing kidney function diagnostic dataset further comprises a base serum creatinine concentration data, the serum creatinine concentration data comprise a first serum creatinine concentration data, a recording date of the base serum creatinine concentration data is the acute kidney injury assessing date, and a recording date of the first serum creatinine concentration data is the closest to the acute kidney injury assessing date among all the serum creatinine concentration data;
wherein the acute kidney injury assessment system further comprises:
a second classifying model for calculating the base serum creatinine concentration data and the first serum creatinine concentration data by the calculating classifier so as to obtain a difference value of serum creatinine concentration, and the difference value of serum creatinine concentration is compared with a threshold of serum creatinine concentration so as to assess a type of AKI of the patient with AKI;
wherein when the difference value of serum creatinine concentration is larger than the threshold of serum creatinine concentration, the type of AKI is a deteriorating type of AKI;
wherein when the difference value of serum creatinine concentration is smaller than the threshold of serum creatinine concentration, the type of AKI is a stable type of AKI.

15. The acute kidney injury assessment system of claim 14, wherein the threshold of serum creatinine concentration is 0.3 mg/dL.

16. The acute kidney injury assessment system of claim 10, wherein the testing kidney function diagnostic dataset further comprises a physiological age data and a gender data, and a risk prognosis data of the result of AKI status is adjusted by the calculating classifier based on the physiological age data and the gender data.

17. The acute kidney injury assessment system of claim 10, wherein the testing kidney function diagnostic dataset further comprises a base glomerular filtration rate data, and a recording date of the base glomerular filtration rate data is the acute kidney injury assessing date;
wherein the acute kidney injury assessment system further comprises:
a kidney care classifying model for analyzing the base glomerular filtration rate data by the calculating classifier so as to obtain a result of kidney care classification.

18. The acute kidney injury assessment system of claim 10, further comprising:
a nephrotoxic drug screening model, wherein the testing kidney function diagnostic dataset further comprises a drug utilization data, and the nephrotoxic drug screening model is for analyzing the drug utilization data so as to output a screening result of nephrotoxic drug usage.
